(19) United States
(12) Patent Application Publication
(10) Pub. No.: US 2011/0054244 A1
(43) Pub. Date: Mar. 3, 2011

(54) Adjustable device for the treatment of female urinary incontinence

(76) Inventors: Bandula Wijay, Friendswood, TX (US); Ayaz Mahmud Durrani, Sugar Land, TX (US); Nandhika Wijay, Friendswood, TX (US); Omar Hayat Durrani, Houston, TX (US)

(21) Appl. No.: 12/584,259

(22) Filed: Sep. 2, 2009

(51) Int. Cl. A61F 2/02

(52) U.S. Cl. 600/30; 600/37

(57) ABSTRACT

A support device suitable for urethral support is adjustable for length post operatively. An elongated body has at least one everting or intersusseception section and a line going through a passage in the elongated body. The elongated body is supported in the patient and the line is adjustable by an adjustable screw. The amount of support can be changed postoperatively using an instrument inserted through the skin with a local anesthetic or even from outside the skin with no surgical intervention. The everting section changes length when adjustments are made.

### FIELD OF THE INVENTION

The field of the invention is a novel device to suspend the female urethra thereby supporting the urethra for the treatment of female stress incontinence and more particularly by using a sling that can be adjusted by utilizing a screw that tightens or loosens a pull string, which regulates the degree to which the urethra is compressed.

### BACKGROUND OF THE INVENTION

Stress urinary incontinence is an involuntary loss of urine on a sudden rise in abdominal pressure as in coughing, sneezing or lifting weight. This type of urinary incontinence occurs when the urethra closes incompletely due to intrinsic deficiency of the muscles of the urethra or hyper mobility of the urethra. Normal urethral muscle function and the normal anatomical contour of the urethra are altered due to over stretching of the pelvic organs and structures during vaginal deliveries. Obesity also brings about similar changes in the urethra. Diabetes causes deterioration of the nerves and muscles of the urethra. Chronic cough, due to smoking or other lung conditions, is also a risk factor in causing stress incontinence of urine. Stress urinary incontinence varies in the degree of loss of control ranging from an occasional leakage of a few drops of urine to a severe loss of urine wherein several diapers are needed per day. Most of the time, this type of incontinence interferes with the normal social life of those affected. In severe cases, those afflicted with stress urinary incontinence incur large expenses for diapers.

Stress urinary incontinence is rare in men. Most commonly, male stress urinary incontinence occurs when the muscles and nerves of the urethra are injured, which may result from radical prostatectomy for the cancer of the prostate.

Treatment of stress urinary incontinence can be attempted with non-surgical methods. For example, the muscles of the urethral sphincter can be strengthened by Kegel exercises. Dr. Arnold Kegel, a gynecologist, designed exercises to strengthen the pelvic floor muscles that impact the urethra. These exercises may be helpful in early cases or could strengthen the muscles before the symptoms develop. A vaginal pessary, which consists of a stiff ring, can be placed in the vagina to press against the urethra to prevent leakage until the bladder contracts to expel the urine. However, a pessary is often not easy for the patient to insert or remove. As a result, a physician usually inserts the pessary into the patient. In addition, a pessary can induce vaginal infection.

Surgical treatment is the mainstay for stress incontinence of urine. In some cases, the walls of the urethra can be approximated by injecting a substance called contigen via a cystoscope. Contigen is a thick gel made from bovine tissue and acts like a bulking agent to approximate the walls of the urethra. However, because it is a foreign material, the patient has to undergo an allergy test for it. After it is injected, contigen has the tendency to gradually leak into the surrounding tissues, necessitating further injections. Besides contigen, other bulking materials are also available. However, these bulking agents are successful in only a very few cases.

The main aim in all surgical procedures for stress urinary incontinence is to give support under the urethra against which the urethra is compressed when abdominal pressure suddenly rises as in coughing, sneezing or lifting heavy objects. In Marshall Marchetti Krantz urethropexy and in Birch urethropexy, the procedures are carried out through an abdominal approach. These procedures are invasive and have a long recovery time. A laparoscopic approach through the abdomen has also decreased in popularity.

Currently, most surgical procedures for the treatment of stress urinary incontinence involve the implantation of a sling. These procedures have some variations, but all are comparatively minimally invasive surgeries. The sling procedures involve placing a sling under the urethra in order to support and compress it so that leakage of urine is prevented during a sudden rise in the abdominal, and consequently the bladder, pressure during coughing, sneezing or lifting heavy objects. The sling can be composed of various materials such as autologous rectus fascia, porcine skin, homologous fascia, and synthetic compounds. There are some variations in the approaches to sling placement. For instance, the sling can be placed through a supra-pubic approach, a vaginal approach, or an obturator approach. As discussed earlier, the most common method for the treatment of female urinary incontinence is to compress the urethra using an implantable sling. In the past, slings have been fixed or anchored in different ways within the abdominal region. The most common anchoring method is to suspend the sling from the pubic bone.

Benderer et. al in U.S. Pat. No. 5,836,314 discloses a procedure for treating female stress incontinence. In his method, Benderer suggests the use of suspension sutures to sling the urethra, and one or more bone anchors in the pubic bone to suspend the sutures.

Most of the traditional surgical methods for implantable and non-implantable devices are very complicated and cumbersome to use. Any surgical intervention, especially in the pelvic region, is very complicated, requiring long recovery and ambulation.

Bouttier in U.S. Pat. No. 7,229,404 B2 describes a device for suspending the urethra using a sling and anchors.

Smith et. al. in U.S. Pat. No. 7,261,723 B1 describes a device sling apparatus that is delivered using a needle handle from below the urethra. The device is also suspended from above to provide the compression on the bladder neck.

Neisz et. al in U.S. Pat. Nos. 7,083,568 and 6,652,450, and Staskin et al. in U.S. Pat. No. 6,612,977 describe a sling wherein the sling apparatus can be permanently changed during and after implantation.

Pretorius et. al. in U.S. Pat. No. 6,786,861 B1 describes a method by which a distensible member is inserted dorsally between the urethra and the pubic bone. By inflating the distensible member via a valve, the distensible member constricts the urethra.

Many patented adjustable slings for urinary incontinence use an expandable element to tighten or loosen the tension of the sling. For instance, Burton et al. in U.S. Pat. No. 7,395,822 describe an adjustable sling that can be post-operatively adjusted through the use of one or more inflatable balloons. Filling or removing material from the balloons is accomplished through the use of a hypodermic syringe. Burton et al. in U.S. Pat. Nos. 7,014,606, 6,579,224, and 6,419,624, describe an adjustable device for urinary incontinence which is adjusted through the use of a fluid expandable element. The device in these patents includes an elongate body which contains the expandable element. The expandable element is accessed via a port located under the patient's skin. Burton et al. in U.S. Pat. No. 6,045,498 and Cook et al. in U.S. Pat. No. 5,964,806 describe an implantable device that includes an expandable element. The expandable element is attached to a conduit through which material is injected or removed post-operatively. Cook et al. in U.S. Pat. Nos. 6,645,138, and 6,419,701, describe an adjustable device for urinary incontinence that expands or contracts by filling or removing fluid from a chamber. The fluid in the chamber is adjusted through the use of a hypodermic syringe. Gil-Vernet in U.S. Pat. No. 6,117,067 describes a sling that can be adjusted after implantation through the use of an expandable chamber. A hypodermic needle extracts or injects fluid into a capsule which is located just beneath the skin and which is connected to the expandable chamber, causing threads that are attached to the expandable chamber to loosen or tighten the sling. Wagner et al. in U.S. Pat. Application No. 20070049790 describe an adjustable sling for urinary incontinence that includes an expandable supportive chamber. The chamber can be filled with gas, fluid, or gel.

Karram et al. in U.S. Pat. No. 6,095,969 describe a fluid-filled urinary incontinence control device that is actuated by abdominal pressure. The device automatically adjusts to changes in abdominal pressure via a diaphragm mechanism.

Li in U.S. Pat. Application No. 20070015957 describes a sling for urinary incontinence that can be adjusted after implantation. The tension on the sling in this invention is adjusted via a removable or biodegradable connection mechanism. Once the connection is removed or dissolved, the tension on the sling automatically adjusts within the patient as scar tissue forms.

Lund et al. in U.S. Pat. Application No. 20040249396 describes a sling for urinary incontinence that can be adjusted post-operatively by removing all or some of the tensioning members of the sling.

Fierro in U.S. Pat Application No. 20040231678 describes a sling that can be adjusted after implantation by displacing the two ends of the sling.

In almost all cases, slings made from a biocompatible material, such as polypropylene, have been used to suspend the bladder neck. Various means to suspend the sling have also been disclosed by many inventors.

Invariably, the sling is hung from either the abdominal wall or from the pelvic bone. Various attachment methods also have been disclosed. In spite of the many inventions, and the devices that materialized subsequently, female stress incontinence is still essentially unresolved, proving that a simple solution to this annoying problem is still needed. In light of the fact that the physiology of the ailment changes post operatively, it is essential that one would consider a device that can adjust the degree to which the urethra is pinched post operatively, and after few weeks or months.

The present invention discloses such methods wherein the device can be adjusted by a screw which tightens or loosens a pull string within a sling, compressing or decompressing the bladder neck and/or urethra.

The major problem with all the previous sling procedures is that there is no control on the amount of tension they place on the urethra. The surgeon only guesses the amount of tension he should leave in the sling. During surgery, the patient is usually under general anesthesia. The body is completely relaxed. Therefore, during the operation, the tension on the sling is different than when the patient is no longer under anesthesia. Also, during surgery, the patient is lying down, and the position of the bladder and the urethra is different as opposed to when the patient is standing up or walking and functioning normally. In the first few days after surgery, there is some edema and swelling due to the surgical trauma. The tension of the sling is comparatively higher during this period, and once the swelling subsides, the sling tension may loosen, reducing its support of the urethra Once the sling is placed and the surgery is completed, there is no way to alter the tension on the sling, resulting in a considerable failure rate in these types of surgeries. As one would expect, there are some cases where the sling is too loose and the surgery has not succeeded in alleviating the incontinence. In other cases, the sling is too tight and the patient cannot urinate. If the sling is too tight, the patient would need catheterization to empty the bladder until another surgery is performed to loosen the sling.

The amount of the pressure exerted by the sling on the urethra can be a significant issue. If the degree of pressure is low, the incontinence is not cured. On the other hand, if the pressure is high, the urethra would not allow urine to pass on active voluntary contraction of the bladder. The delicate balance in the degree of compression needed to achieve normal urination cannot be achieved through guessing or trial and error. Presently, none of the procedures for the treatment of stress urinary incontinence have any control on the degree of compression placed on the urethra. The novel device presented here is the only device where the compression placed on the urethra is adjustable.

In addition, clinical evidence has shown that the current procedure of anchoring the sling to the pubic bone can cause infection resulting in osteitis pubis. The suspension of the sling on the pubic bone with metallic or non-metallic screws, and similar fasteners, has resulted in other complications, as the screws are not very stable, and can even detach from the bone. As such, one aspect of the present invention recommends that as the surrounding tissue grows into the fenestrations of a mesh made from polypropylene, or similar material, the sling will be sufficiently anchored within the body.

### SUMMARY OF THE INVENTION

The present invention consists of a device that supports the bladder neck and urethra and is minimally invasive to implant. It comprises:
1. A sling to support the urethra;
2. A pull string attached to a screw within the sling;
3. An everting section that expands or contracts as the screw and pull string are loosened or tightened; and
4. A removable or implanted tube through which the screw can be accessed externally.

The proposed sling, unlike slings that have been disclosed in the past (which are mainly polypropylene mesh devices that are often distensible), is preferably a flexible and non-distensible thin-walled tube. The tube preferably has a 3-4 mm. diameter. As the tube is thin-walled, when used as a sling, it will take the shape of a flat belt. The sling tube is typically made from nylon, polyester, irradiated polyethylene, or polypropylene. The tube is made from a bi-axially oriented polymer such that the tube is preferably neither able to expand in diameter or length when a circumferential or lengthwise force is applied. The sling can be silicone coated to minimize tissue adhesion.

The non-distensible character of the sling prevents, or minimizes, stretch and yield with time due to the abdominal pressure or urge pressure by the patient. Therefore, the sling will not appreciably relax with time and will maintain its ability to compress the urethra as the patient performs normal everyday activities.

The sling has an everting section which allows the sling to loosen or tighten against the urethra or bladder neck as the pull string is adjusted from a screw that is positioned within the sling device. The everting section also allows the surgeon to adjust the sling length during, and after, the procedure.

The sling has a non-stretchable pull string attached at both ends which is preferably made from woven polyester, nylon, or silk, and which performs best when it is made into a flat ribbon. The pull string is placed inside the sling tube and is anchored at the distal end with a knot. At the proximal end, the pull string is attached to an externally accessed adjustable screw. When the screw is tightened, the pull string tightens, contracting the everting section, causing the sling to shorten and thereby compressing the urethra. Similarly, loosening the screw loosens the pull string, relaxing the everting section, allowing the sling to lengthen, enabling the urethra to decompress.

Thus, the sling is able to provide compression on the urethra by applying tension on the pull string, which is inside the sling tube. The tension on the pull string provides the means to adjust the degree to which the sling compresses the urethra.

The sling components are preferably non stretchable and non distensible to prevent creep and yield after the implantation of the sling.

The anatomy of each patient is different, requiring slings of different lengths This problem is resolved during surgery by selecting a sling of the correct length.

In summary, the present invention comprises a preferably non-distensible urethral sling for the treatment of female stress urinary incontinence which can be adjusted in order to compress or relax the urethra. This functionality is accomplished by tugging on a pull string, which is located within the sling sheath. The pull string is anchored at the distal end with a knot. The proximal end of the pull string is attached to a screw that is situated inside the sling. The screw adjusts the tension on the pull string which impacts the everting section of the sling causing the sling to compress or decompress the urethra. The screw is accessed externally through a removable tube that extends through the skin of the patient. After the sling is adjusted, the removable tube is replaced with a permanent cap.

Therefore, this invention allows the surgeon to adjust the degree to which the sling compresses the urethra during and immediately after surgical implantation, or many weeks or months after surgery.

### BRIEF DESCRIPTION OF THE DRAWINGS

FIG. 1 shows a diagrammatic anatomic view of the pelvic region of the human female and the general method of the introduction of a needle to pull a urethral sling;

FIG. 2A shows a diagrammatic view of the proposed sling;

FIG. 2 B shows a diagrammatic view of the proposed sling when the adjusting Module is outside of the patient's body;

FIG. 3 shows a diagrammatic view of the adjusting screw;

FIG. 4 shows the implanted sling with the removable tube replaced with a permanent cap;

FIG. 5 is a special coaxial screwdriver for adjusting the sling.

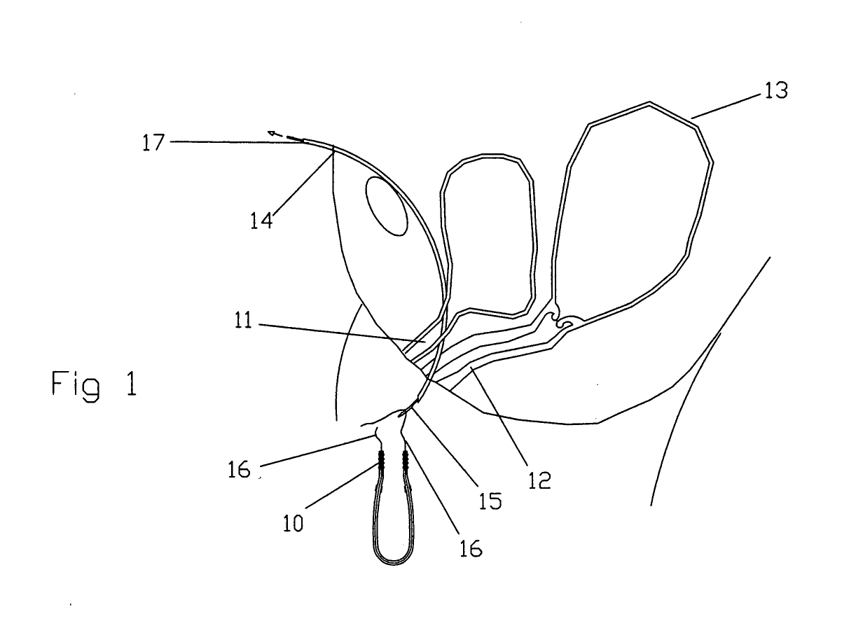

### DETAILED DESCRIPTION OF THE PREFERRED EMBODIMENT FIG. 1 is an anatomic description of the pelvic region of the human female. The proposed urethral sling 10 is introduced bilaterally just below the urethra 11 in the space between the urethra 11 and the vagina 12 using a curved needle 14 introduced anteriorly and behind the pubic bone on either side of the urethra 11. Typically, the surgeon creates a pocket between the urethra 11 and vagina 12 where the sling 10 will be placed. The needle 14 can be made blunt or sharp. If it is made sharp, the physician must wear a thimble on his finger to receive the needle 14 when it is pushed from above. When the needle 14 is made hollow, it enables the operator to thread a loop guide wire 15 to facilitate threading of the pull thread 16 through the needle. The thread 16 is pulled through the needle 14 along with the sling 10 until one end of the sling 10 exits the skin at the location 17 where the needle 14 was introduced. The same process is repeated so that the other end of the sling 10 is pulled on the opposite side of the urethra 11 in the same manner until the sling 10 and the pull thread 16 exits the skin at which place the needle 14 entered.

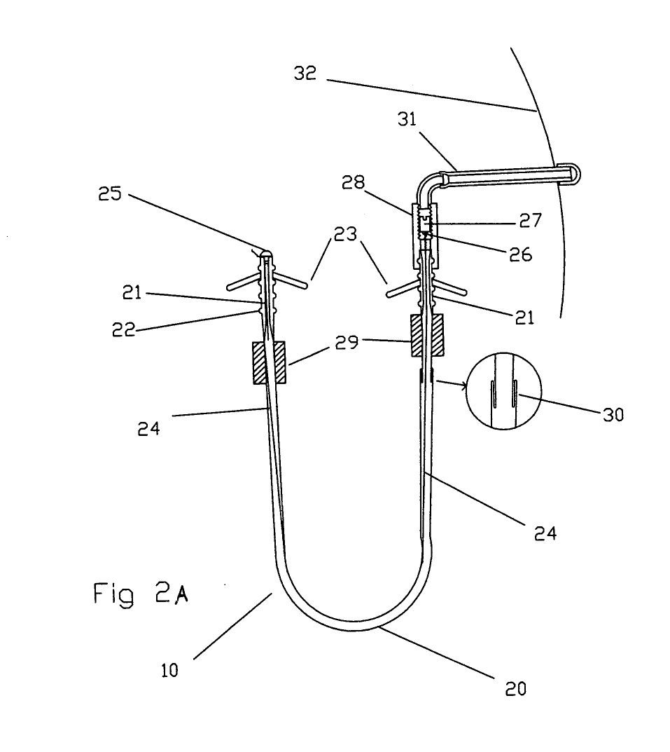

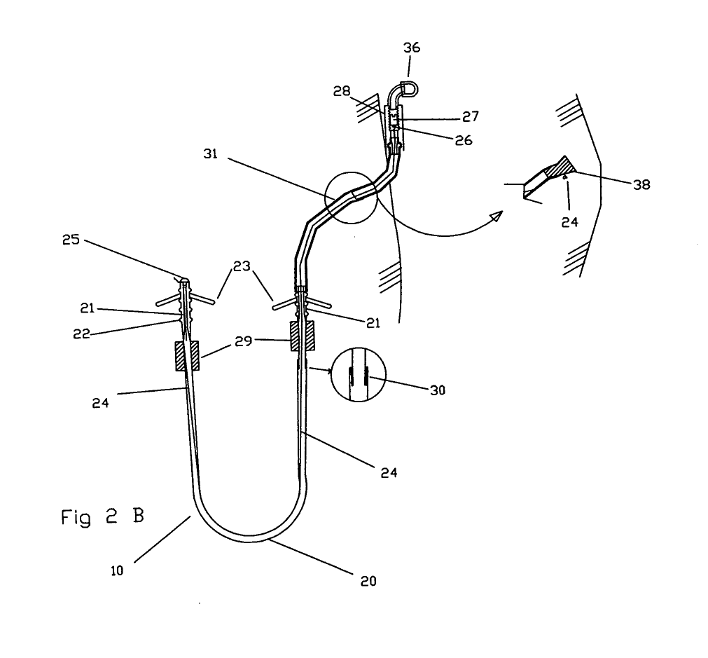

FIG. 2A is a diagrammatic representation of the sling 10. The sling 10 consists of a tubular portion 20 that is made from a thin wall plastic material such as Nylon, Polyester (PET), Polyester (PBT), Polyethylene, Polypropylene, or other suitable material. The sling 10 is an elongated tube that is made from a bi-axially or uni-axially oriented material in order to impart strength. The sling 10 can also be made from Silicone rubber, or braided, or fabric reinforced, Silicone rubber. The sling 10 has a neck region 21, which is an elongated tube structure containing “barbs” 22. The barbs 22 are circular in nature, or they can also be protrusions that are segments of a circular ring. The barbs 22 can be rounded as a collar or teeth like. Additionally, two or three anchors 23 may also be provided which increase tissue adhesion of the sling neck 21. The pull member 24 has the preferred shape of a ribbon and is placed within the sling 10. This pull member 24 is made from a strong material such as molecular oriented material including Nylon, Polyester, Polypropylene or multithreaded silk, or suture materials such as catgut. The pull member 24 is used to pull or relax on the sling 10 to constrict or relax the urethra 11. The distal end of the pull member 24 is anchored to the neck region 21 with a knot 25. The proximal end of the pull member 24, which lies within the elongated tube, is threaded through a hole 26 of a screw 27, which is in a threaded tube 28, the “adjusting module” that is attached to the contra lateral end of the sling tube 10 to the neck 21. The threaded tube 28 is provided with appropriate screw threads that will accommodate the screw 27. The screw 27 can be made from nylon or stainless steel or any suitable material. In an alternate design, the adjusting module 28 can also be an integral part of the sling neck 21. The sling 10 also contains an everting or intersusseception section 30, which expands or contracts as the tension on the pull member 24 is released or tightened. When the pull member 24 is tightened by turning the screw 27, the pull member 24 causes the everting section 30 to contract or collapse, thereby increasing the tension on the sling 10 and on the urethra 11. When the pull member 24 is relaxed by loosening the screw 27, the everting section 30 expands, loosening the tension on the sling 10 and on the urethra 11. Attached to the “adjusting module” 28 end of the sling 10 is a tube 31 which can be made from polyurethane or silicone and can be made kink-free by either using a braiding or a spiral reinforcement within the walls or adjacent to the walls of the tube 31. The tube 31 is attached to the sling 10 adjusting module 28 just proximal to the screw 27. The tube 31 can also be made to be an integral part of the sling 10 and hence the “adjusting module” 28. The tube 31 extends through the skin 32 and is used to provide external access to the head of the screw 27. The tube 31 may be removed at a later time, after the swelling has subsided and after the final adjustment has been performed to adjust the sling 10 tension, simply by pulling on it, or by a quick “tug” to separate it from the adjusting module 28. A polypropylene mesh 29 is attached near the proximal and distal ends of the sling 10, in the sling neck region 21, which further help to anchor the sling 10 to the surrounding tissue. The screw 27 is adjusted using a screwdriver having a flexible shaft introduced through the tube 31.

FIG. 2 B shows the sling in FIG. 2A except that the adjusting module is outside the patient's body. The adjusting module 28 is connected to the sling neck 21 with a tube 31. The adjusting module can be removed from the tube 31 and capped off by cap 36 after making a final adjustment. Additionally the tube can be cut off just under the skin so that after capping off the tube 31 with cap 36, the skin incision can be closed off by suture. In order to secure the pull member 24 in the taught position a plug 38 can also be used before capping off with the cap 36. This will maintain the tension on the sling after the adjusting module has been removed.

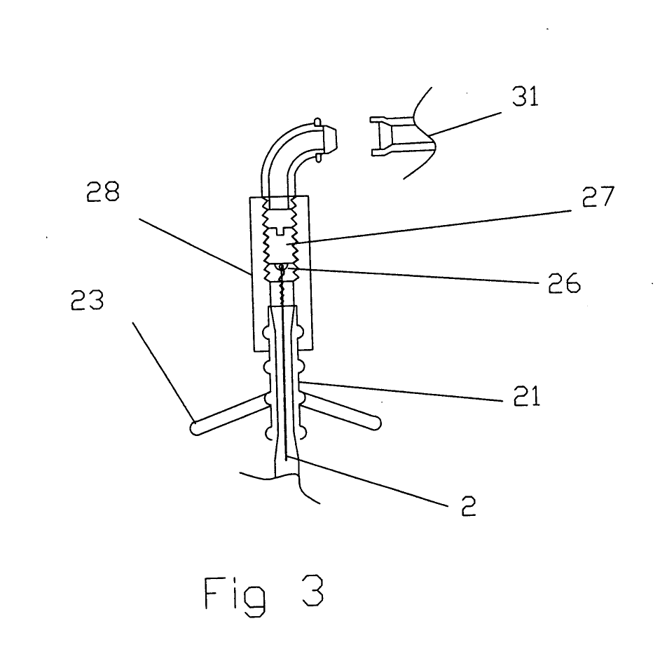

FIG. 3 shows the adjusting module 28 which is located adjacent to the neck region 21 of the proximal end of the sling 10. The adjusting module 28 is threaded so that the screw 27 turns inside the adjusting module 28. The thread in the tube 28 can also be a spring coil, acting as a thread (not shown) of appropriate pitch embedded in the material of the tube 28, and hence making the tube 28 flexible. The screw 27 can be made from nylon or stainless steel and contains a hole 26. The pull member 24 is threaded through the hole 26 of the screw 27. The tube 31 can be detachably attached to the adjusting module 28.

Within a few weeks after the surgery, and after the edema has subsided, the surgeon can, under local anesthesia, adjust the tension of the pull member 24 by turning a screw 27 using a screw driver introduced through a temporary tube 31 to turn it either direction to tension or relax the pull member 24.

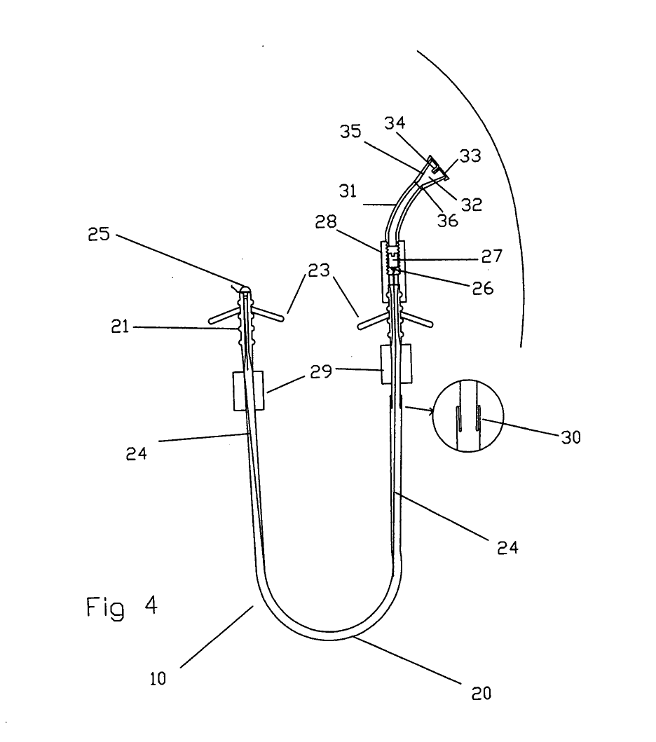

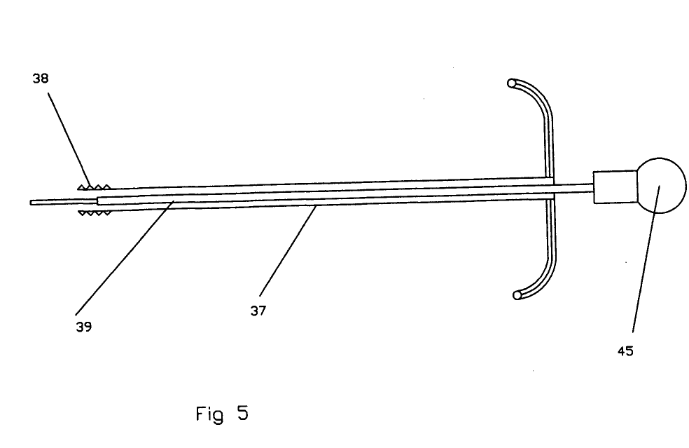

FIG. 4 shows the implanted sling 10 with an alternate design where the tube 31 terminates within the body below the skin. The tube is provided with a screwdriver receptacle 32, which is capped with a silicone rubber cap 33. The rubber cap 33 is of molded rubber such that the center of which consists a valve feature 34 that allows the screwdriver to enter but will seal once the screwdriver is removed. The receptacle has a tapered throat 35 to guide the screwdriver “needle” to the center and to the interior of the tube 31 thereby facilitating the screwdriver to engage with the slot in the screw head in the screw 27. The thread 36 in the throat of the receptacle 32 enables a special tool in FIG. 5 to engage such that the receptacle 32 is held in place during the adjusting process, so that the axial pressure during the adjustment does not push the receptacle further into the body cavity.

FIG. 5. Shows a special coaxial screwdriver having an outer casing 37 with a male thread feature 38 at its distal end with a flexible central shaft 39 containing a slotted screwdriver head. The casing 37 is introduced through the skin and the receptacle 32 is engaged by screwing the male thread 38 into the female thread 36 in the receptacle 32. The thread style, male/female can also be reversed depending on the exact design of the receptacle 32. The flexible screwdriver shaft 39 is then introduced through the needle casing 37 until the tip of the screwdriver engages with the screw 27. By turning the screwdriver 39, the sling can be adjusted to the desired tension.

The above description of the device invented here is an introduction of the concepts. No effort was made to provide any engineering details of construction and it is not our intension to provide such details. As such, this prosthesis can be redesigned to appear different and can be made from a variety of bio-compatible materials. The advantage of the device is its ability to adjust the sling 10 tension, with the preferred techniques described above, immediately after, or a few days or weeks after the surgery, which is not possible in the various previous inventions and products. The sling 10 in the above invention will not attach to the tissue due to its smooth surface and will enable the surgeon to adjust the tension. In addition, it has an everting section 30 that allows adjustment of the sling 10 during and or after sling implantation. The pull member 24 is located inside the sling tube 10 and is not in contact with body tissue. As such the pull member 24 will not form “adhesion” with the body tissue allowing free movement required for adjustment.

The operation uses two curved needles 14 that are introduced through a small incision in the skin just above the pubic bone and exit on either side of the urethra 11 in the space between the urethra 11 and the wall of the vagina 12. The invention discussed here proposes a hollow needle with a sharp obturator to facilitate the process of pushing the needle, as discussed above. It is important that the physician wear a thimble in his gloved finger to receive the needle.

The device discussed above will enable the female to control the urinating function and will be an excellent solution to the problem of incontinence faced by millions of patients worldwide.

The above description is illustrative of the preferred embodiment and many modifications may be made by those skilled in the art without departing from the invention whose scope is to be determined from the literal and equivalent scope of the claims below.

We claim:

1. An implantable medical device to provide internal organ support for a patient, comprising:
an elongated body adapted to internally support an internal organ and having thereon a pair of fixation locations to accomplish said support;
a length adjustment feature associated with said body to facilitate post operative length adjustment to optimize the function of the internal organ.

2. The device of claim 1, wherein:
said elongated body is tubular and said adjustment feature is integral to said elongated body.

3. The device of claim 2, wherein:
said adjustment feature comprises at least one everting segment in the wall of said elongated body.

4. The device of claim 1, wherein:
said elongated body is tubular and said adjustment feature comprises a line extending through a passage in said body.

5. The device of claim 4, wherein:
said line is post operatively movable relative to said fixation locations from outside or inside the patient's body.

6. The device of claim 4, wherein:
movement of said line changes the length of at least one bellows in the wall of said body.

7. The device of claim 4, wherein:
said line is connected to a movable member in an adjustment housing with said adjustment housing providing access to said movable member from a location exterior to said housing.

8. The device of claim 7, wherein:
movement of said movable member changes the length of said everting section.

9. The device of claim 8, wherein:
said movable member is rotated

10. The device of claim 7, wherein:
said adjustment housing is flexible.

11. The device of claim 10, wherein:
said housing is made using a spring that provides threads on which said movable member moves axially when rotated.

12. The device of claim 7, wherein:
said tubular elongated body comprises a neck portion at opposed ends with an anchoring feature.

13. The device of claim 12, wherein:
said anchoring feature comprises a plurality of protrusions of differing heights.

14. The device of claim 12, wherein:
said adjustment housing is connected to one of said neck portions.

15. The device of claim 9, wherein:
said adjustment housing comprises an access tube connected adjacent an end thereof.

16. The device of claim 15, wherein:
said access tube is removably mounted.

17. The device of claim 16, wherein:
said access tube extends outside the skin of the patient.

18. The device of claim 15, wherein:
said access tube further comprises a valve that admits a tool therethrough for rotation of said movable member and closes after the tool is removed.

19. The device of claim 18, wherein:
said access tube does not extend outside the skin of a patient.

20. The device of claim 15, wherein:
said movable member is accessed with a tool having a rotatable member in a sheath said tool inserted through said access tube whereupon said sheath removably attaches to said adjustment housing when said rotatable member is engaged to said movable member.

21. The device in claim 1, where the adjustment feature is located outside the patient's body.

22. The device in claim 21 where the adjustment module is removable after adjustments.

23. The device in claim 22 where the pull string is plugged after adjustment to maintain the adjusted tension.
